(19) United States
(12) Patent Application Publication
(10) Pub. No.: US 2006/0019987 A1
(43) Pub. Date: Jan. 26, 2006

(54) Methods and compositions for inhibiting, destroying, and/or inactivating viruses

(76) Inventors: Charles A. Fust, Englewood, FL (US); Kevin S. Harrod, Albuquerque, NM (US); Adriana E. Kajon, Albuquerque, NM (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/187,575

(22) Filed: Jul. 22, 2005

Related U.S. Application Data
(60) Provisional application No. 60/590,781, filed on Jul. 23, 2004.

(51) Int. Cl. A61K 31/47; A61K 31/44; A61K 31/14

(52) U.S. Cl. 514/307; 514/358; 514/311; 514/643

(57) ABSTRACT

The present disclosure provides compositions, methods, and processes for the inhibiting, destroying, and/or inactivating viral contaminants in a biological source material, or treatment of viral infections. The disclosed compositions include one or more quaternary ammonium compounds. One exemplary method includes contacting the biological source material with a solution containing one or more quaternary ammonium salts.

### CROSS-REFERENCE TO RELATED APPLICATION

This application claims priority to copending U.S. provisional application titled, “Methods and Compositions for Inhibiting, Destroying, and/or Inactivating Viruses,” having Ser. No. 60/590,781, filed Jul. 23, 2004, which is entirely incorporated herein by reference.

### BACKGROUND

1. Technical Field

The present disclosure is generally related to methods, compositions, or processes for inhibiting, destroying, and/or inactivating viruses present in host organisms or samples/process streams of biological origin.

2. Related Art

Virus caused respiratory illnesses account for much of the suffering and inconvenience endured by mankind and animals generally, and, in some instances, account for high rates of mortality. For example, influenza is one of the common diseases of man, infecting large segments of the population each year, typically during the fall and winter and early spring of the year, with great economic consequences and, occasionally, with great public health consequences. Notwithstanding that influenza has been extensively studied, very little progress has been made toward the prevention or cure of the disease. One reason for the slow progress toward preventing or treating influenza is the antigenic shift which presents frequent and often abrupt appearances of new serotypes with the consequence that an inactivated virus vaccine against one serotype may have little or no immunizing effect against other serotypes.

Members of the family paramyxoviridae are responsible for a number of serious diseases in humans and animals. Bronchiolitis is one of the most serious pulmonary infections commonly caused by respiratory syncytial virus (RSV), a member of the paramyxoviridae. RSV disease occurs in yearly epidemics and is most severe in children 1 year of age or younger. Approximately 1 in 50 to 1 in 100 infants are hospitalized after their first infection, and mortality fluctuates between 0.5 and 5.0 percent. Patients with underlying conditions such as congenital heart disease and bronchopulmonary dysplasia are at higher risk for morbidity and mortality. RSV disease has also been documented in immunocompromised adults, aged 21 to 50, where the immune system had been compromised by bone marrow transplants, renal transplants, pancreas transplants and by T-cell lymphoma, based on specimens from bronchoalveolar lavage, sputum, throat, sinus aspirate, and lung biopsy. (Respiratory syncytial virus infection in immunocompromised adults, Englund J A; Sullivan C J; Jordan M C; Dehner L P; Vercellotti G M; Balfour H H Jr, Ann. Intern. Med., Aug. 1, 1988, 109 ( 3) p.203-8.) Further, RSV is most well known as the causative virus responsible for the common cold.

With respect to the adenovirus family, there are over 40 different adenovirus varieties, some of which cause the common cold. Adenovirus is of major concern to the military for new recruits living in confined quarters. It is responsible for the hospitalization and resultant retraining of these recruits. The Center for Disease Control (CDC) and The National Institute of Health (NIH) are concerned about adenovirus with civilian populations in confined settings such as hospitals, schools, and institutions. No vaccine is currently available for the adenovirus.

Pneumonias in adults due to mycoplasma, chlamydiae, and viruses are a common clinical problem. These microorganisms contribute to the etiologies in 6-35% of all cases of pneumonia and are the sole pathogens in 1-17% of hospitalized cases. Important trends and developments in the field include the emergence of a Chlamydia psittaci strain (TWAR) that is passed from human to human, causes a mycoplasma-like illness, and is relatively resistant to erythromycin, the recognition of respiratory syncytial virus as a pathogen in nursing home outbreaks and in immunosuppressed adults, the continuing high lethality of fully developed influenza pneumonia, the efficacy of acyclovir and adenine arabinoside in limiting the complications of varicella-zoster virus infections, and the increasing frequency of pneumonia caused by cytomegalovirus and the severity of this disorder in highly immunosuppressed patients.

Cytomegalovirus (CMV) pneumonia causes significant morbidity and mortality in bone marrow transplant recipients and in patients with AIDS. 9-(1,3-Dihydroxy-2-propoxymethyl) guanine (ganciclovir) and phosphonoformic acid (PFA) demonstrate activity against CMV in human infections, although recurrent CMV and systemic drug toxicity frequently develop. The efficacy of aerosol administration of antiviral agents against murine CMV (MCMV) infection has been examined using aerosolized ganciclovir, PFA, or ribavirin. The results suggest that aerosol administration of antiviral agents can potently and selectively inhibit replication of MCMV in the lung. (Aerosol administration of antiviral agents to treat lung infection due to murine cytomegalovirus, Debs R J; Montgomery A B; Brunette E N; DeBruin M; Shanley J D, J. Infect. Dis. (UNITED STATES) February 1988, 157 (2) p.327-31.)

Progress is, however, being made in the development of drugs for the prevention and treatment of viral infections, as opposed to only using vaccines as a preventative measure. Two drugs currently available to clinicians are amantadine (Symmetral™) and ribavirin (Virazol®). Oral amantadine is effective for both treatment and prevention of uncomplicated influenza A infections. Ribavirin aerosol is now used with some success in the treatment of RSV infections. (Ribavirin aerosol treatment of serious respiratory syncytial virus infection in infants, Rodriguez W J; Parrott R H, Infect. Dis. Clin. North Am., (UNITED STATES) June 1987, 1 (2) p.425-39). Although vaccination continues as the mainstay of influenza prevention, antiviral drugs are useful for unvaccinated patients if complications are likely or if vaccines do not exist or are not plausible to administer. It is apparent from the foregoing discussion there remains a need for improved methods of inhibiting, destroying, or inactivating pathogenic viruses in host organisms and in other biological sources.

### SUMMARY

Aspects of the present disclosure are generally directed to compositions and methods for the treatment of viral pathologies. One aspect of the present disclosure is directed to compositions that include at least one quaternary ammonium salt that is used to inhibit, destroy, or inactivate viruses. Exemplary common viruses that can be treated according to the present disclosure include, but are not limited to, Respiratory Syncytial Virus (RSV), Adenovirus, Severe Acute Respiratory Syndrome (SARS) virus, and small pox.

Another aspect of the disclosure provides methods for treating viruses in biological source material or host organisms. In one example, the method includes contacting a biological source material with a composition that includes a quaternary ammonium salt. In another example, the disclosure relates to treating an organism infected by a virus with a composition that includes a quaternary ammonium salt.

### BRIEF DESCRIPTION OF THE DRAWINGS

The accompanying figures, which are incorporated in and form part of the specification, further illustrate the disclosed compositions and methods and, together with the detailed description, serve to explain the principles of the present disclosure.

FIG. 1 depicts micrographs that illustrate the reduction of hAd4 virus infectivity in A549 human lung epithelial cells by the disclosed compositions and methods.

FIG. 2 depicts micrographs that illustrate the reduction of hAd5 virus infectivity in A549 human lung epithelial cells by the disclosed compositions and methods.

FIG. 3 depicts micrographs that illustrate the reduction of RSV virus infectivity in Hep-2 human lung epithelial cells by the disclosed compositions and methods.

FIG. 4 depicts electron micrographs of hAd4 viral particles treated with exemplary disclosed compositions.

### DETAILED DESCRIPTION

1. Definitions

Unless otherwise indicated the following terms used in the specification and claims have the meanings discussed below:

The term “organism” refers to any living entity comprised of at least one cell. A living organism can be as simple as, for example, a single eukaryotic cell or as complex as a mammal, including a human being.

The term “biological source material” refers to any biological material such as, for example, a host cell, cell supernatant, cell lysate, blood plasma, tissue homogenate, or other biological materials.

The term “therapeutically effective amount” as used herein refers to that amount of the compound being administered which will relieve to some extent one or more of the symptoms of the disorder being treated. In reference to viruses, a therapeutically effective amount refers to that amount which has the effect of (1) reducing the amount of any virus, (2) inhibiting (that is, slowing to some extent, preferably stopping) any virus, (3) inducing the growth or viability of immune system cells that fight viruses, and/or, (4) relieving to some extent (or, preferably, eliminating) one or more symptoms associated with the any viral related disease.

“Pharmaceutically acceptable salt” refers to those salts which retain the biological effectiveness and properties of the free bases and which are obtained by reaction with inorganic or organic acids such as, but not limited to, hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or pharmaceutically acceptable salts thereof, with other chemical components, such as physiologically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound. “Carriers” as used herein include pharmaceutically acceptable carriers, excipients, or stabilizers which are nontoxic to the cell or mammal being exposed thereto at the dosages and concentrations employed.

An “excipient” refers to an inert substance added to a pharmaceutical composition to further facilitate administration of a compound. Examples, without limitation, of excipients include calcium carbonate, calcium phosphate, various sugars and types of starches, cellulose derivatives, gelatin, vegetable oils, and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to viral infections, these terms simply mean that the viral pathogens are reduced, eliminated, inactivated, or that one or more of the symptoms of the disease will be reduced.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962) “Drug Latentiation” in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APHA; H. Bundgaard, ed. (1985). Design of Prodrugs, Elsevier; Wang et al. (1999). Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990). Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985). Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H.K. et al. (2000). Targeted prodrug design to optimize drug delivery, AAPS PharmSci., 2(1): E6; Sadzuka Y. (2000). Effective prodrug liposome and conversion to active metabolite, Curr Drug Metab., 1(1):31-48; D. M. Lambert (2000). Rationale and applications of lipids as prodrug carriers, Eur. J Pharm. Sci., 11 Suppl 2:S15-27; Wang, W. et al. (1999). Prodrug approaches to the improved delivery of peptide drugs. Curr. Pharm. Des., 5(4):265-87. Where applicable, the instant disclosure should be construed to include the prodrug as well as the parent drug or active ingredient.

The term “inhibition” of a unicellular living creature or of a virus means either hindering its proliferation, or making it incapable of accomplishing some functions that it usually accomplishes.

The term “destroying” means killing the unicellular living creatures or viruses.

The term “substance” hereafter means any chemical compound or association of chemical compounds having at least one given function or one function common to the compounds, and which can be included in the composition of a finished product, generally associated with one or more excipients and possibly with other substances. Similarly, the term “product” means a usable finished product. Thus, a finished product is generally constituted of at least one excipient and of several substances, each substance being constituted of one or several chemical compounds having similar or identical functions. The term “substance” may correspond to an actual fact, but may be purely theoretical and functional in the case of intricate mixtures where the compounds have multiple effects or which effects interfere with each other. The functional classification in compounds, substances, products does not necessarily correspond to the process of manufacture of the product and to the mixture actually obtained in the practice.

The term “composition” is used here and in all the following text to define a pharmaceutical or cosmetic substance.

As used herein, the term “alkyl group” is intended to mean a straight- or branched-chain monovalent radical of saturated and/or unsaturated carbon atoms and hydrogen atoms, such as methyl (Me), ethyl (Et), propyl, isopropyl, butyl, isobutyl, t-butyl, ethenyl, pentenyl, butenyl, propenyl, ethynyl, butynyl, propynyl, pentynyl, hexynyl, and the like, which may be unsubstituted (i.e., containing only carbon and hydrogen) or substituted by one or more suitable substituents (e.g., one or more halogens, such as F, Cl, Br, or I, with F and Cl being preferred). A “lower alkyl group” is intended to mean an alkyl group having from 1 to 4 carbon atoms in its chain. Preferred alkyl groups are C₁-C₁₈, more preferably C₈-C₁₀.

An “alkoxy group” is intended to mean the radical —ORₐ, where Rₐ is an alkyl group. Exemplary alkoxy groups include methoxy, ethoxy, propoxy, and the like.

A “cycloalkyl group” is intended to mean a non-aromatic monovalent monocyclic, bicyclic, or tricyclic radical containing 3, 4, 5, 6, 7, 8, 9, 10, 11, 12, 13, or 14 carbon ring atoms, each of which may be saturated or unsaturated, and which may be unsubstituted or substituted by one or more suitable substituents as defined below, and to which may be fused one or more heterocycloalkyl groups, aryl groups, or heteroaryl groups, which themselves may be unsubstituted or substituted by one or more substituents.

A “heterocycloalkyl group” is intended to mean a non-aromatic monovalent monocyclic, bicyclic, or tricyclic radical, which is saturated or unsaturated, containing 3, 4, 5, 6, 7, 8, 9, 10, 11, 12, 13, 14, 15, 16, 17, or 18 ring atoms, which includes 1, 2, 3, 4, or 5 heteroatoms selected from nitrogen, oxygen, and sulfur, where the radical is unsubstituted or substituted by one or more suitable substituents as defined below, and to which may be fused one or more cycloalkyl groups, aryl groups, or heteroaryl groups, which themselves may be unsubstituted or substituted by one or more suitable substituents.

An “aryl group” is intended to mean an aromatic monovalent monocyclic, bicyclic, or tricyclic radical containing 6, 10, 14, or 18 carbon ring atoms, which may be unsubstituted or substituted by one or more suitable substituents as defined below, and to which may be fused one or more cycloalkyl groups, heterocycloalkyl groups, or heteroaryl groups, which themselves may be unsubstituted or substituted by one or more suitable substituents.

A “heteroaryl group” is intended to mean an aromatic monovalent monocyclic, bicyclic, or tricyclic radical containing 4, 5, 6, 7, 8, 9, 10, 11, 12, 13, 14, 15, 16, 17, or 18 ring atoms, including 1, 2, 3, 4, or 5 heteroatoms selected from nitrogen, oxygen, and sulfur, which may be unsubstituted or substituted by one or more suitable substituents as defined below, and to which may be fused one or more cycloalkyl groups, heterocycloalkyl groups, or aryl groups, which themselves may be unsubstituted or substituted by one or more suitable substituents.

An “acyl group” is intended to mean a —C(O)—R radical, where R is a substituent.

A “thioacyl group” is intended to mean a —C(S)—R radical, where R is a substituent.

The term “flavor” or “flavoring agent” as used herein refers to an agent in a form of an emulsion, concentrate, aqueous- or oil-soluble liquid or a dry powder which may be added to the compositions and does not trigger vasomotor rhinitis.

2. Pharmaceutical Compositions

Exemplary embodiments include pharmaceutical compositions that can be manufactured by processes well known in the art, e.g., by means of conventional mixing, dissolving, granulating, dragee-making, levigating, emulsifying, encapsulating, entrapping, lyophilizing processes or spray drying. Moreover, in certain embodiments, the compositions may be formulated for horticultural or agricultural use. Such formulations include dips, sprays, seed dressings, stem injections, sprays, and mists. In some embodiments, the pharmaceutical compositions include as an active ingredient a quaternary ammonium salt in an amount sufficient to inhibit, destroy, or inactivate a virus.

The compositions of the present disclosure can be liquids or lyophilized or otherwise dried formulations and can include diluents of various buffer content (e.g., Tris-HCl, acetate, phosphate), pH and ionic strength, additives such as albumin or gelatin to prevent absorption to surfaces, a surfactant such as a polysorbate surfactant (e.g., TWEEN 20, TWEEN 40, TWEEN 60, and TWEEN 80), a phenoxypolyethoxyethanol surfactant (e.g., TRITON X-100, X-301, X-165, X-102, and X-200, and TYLOXAPOL), Pluronic F68, or sodium dodecyl sulfate, solubilizing agents (e.g., glycerol, polyethylene glycerol), anti-oxidants (e.g., ascorbic acid, sodium metabisulfite), preservatives (e.g., Thimerosal, benzyl alcohol, and parabens), bulking substances or tonicity modifiers (e.g., lactose, and mannitol). Such compositions can also include covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions can influence the physical state, solubility, stability, rate of in vivo release, and rate of in vivo clearance. Controlled or sustained release compositions include the formulation in lipophilic depots (e.g., fatty acids, waxes, and oils).

The present disclosure contemplates formulations that may be employed in pharmaceutical and therapeutic compositions and applications suitable the treatment of viral infections, including, but not limited to, Respiratory Syncytial Virus (RSV), Adenovirus, Severe Acute Respiratory Syndrome (SARS) virus, and small pox. Such compositions may be employed to reduce, inhibit, eliminate, destroy, and/or inactivate viruses.

For in vivo applications, the compositions can be administered using an effective pharmaceutically acceptable form to an organism, including human and animal subjects. Generally, this entails preparing compositions that are essentially free of pyrogens, as well as other impurities that could be harmful to humans or animals.

Other embodiments provide particulate compositions coated with polymers (e.g., poloxamers or poloxamines). Still other embodiments of the compositions incorporate particulate forms, protective coatings, protease inhibitors or permeation enhancers for various routes of administration, including, but not limited to, parenteral, pulmonary, nasal and oral. In one embodiment the pharmaceutical composition is administered buccally, rectally, vaginally, topically, nasally, parenterally, paracancerally, transmucosally, transdermally, intramuscularly, intravenously, intradermally, subcutaneously, intraperitonealy, intraventricularly, intracranially, intratumorally, in the form of a spray or in any other form effective to deliver active compositions.

For topical applications, the pharmaceutically acceptable carrier may take the form of a liquid, cream, foam, lotion, or gel, and may additionally comprise organic solvents, emulsifiers, gelling agents, moisturizers, stabilizers, surfactants, wetting agents, preservatives, time release agents, and/or minor amounts of humectants, sequestering agents, dyes, perfumes, and/or other components commonly employed in pharmaceutical compositions for topical administration.

Further, as used herein “pharmaceutically acceptable carriers” are well known to those skilled in the art and include, but are not limited to, 0.01-0.1M and preferably 0.05M phosphate buffer or 0.8% saline. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g., fatty acids, waxes, oils). Also comprehended are particulate compositions coated with polymers (e.g., poloxamers or poloxamines) and the compound coupled to antibodies directed against tissue-specific receptors, ligands or antigens or coupled to ligands of tissue-specific receptors.

Tablet and dosage forms of the compositions, in which the emulsions are formulated for oral or topical administration, include liquid capsules and suppositories. In solid dosage forms for oral administration, the compositions may be admixed with one or more substantially inert diluents (e.g., sucrose, lactose, starch, and the like) and may additionally comprise lubricating agents, buffering agents, enteric coatings, and other components well known to those skilled in the art.

Compounds modified by the covalent attachment of water-soluble polymers, such as polyethylene glycol, copolymers of polyethylene glycol and polypropylene glycol, carboxymethyl cellulose, dextran, polyvinyl alcohol, polyvinylpyrrolidone or polyproline, are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref Biomed Eng 14:201; Buchwald et al. (1980). Surgery 88:507; Saudek et al. (1989). N. Engl. J Med. 321:574). In another embodiment, polymeric materials can be used. In yet another embodiment, a controlled release system can be placed in proximity of the therapeutic target, i.e., the lungs, thus requiring only a fraction of the systemic amount. Preferably, a controlled release device is introduced into a subject in proximity of the site of a viral infection. Other controlled release systems are discussed in the review by Langer (1990). Science 249:1527-1533.

In other embodiments, the compositions may be impregnated into absorptive materials, such as sutures, bandages, and gauze, or coated onto the surface of solid phase materials, such as surgical staples, zippers and catheters to deliver the compositions to a site for the prevention of viral infection. Other delivery systems of this type will be readily apparent to those skilled in the art.

Examples of suitable oily vehicles or solvents for use with the present disclosure are vegetable or animal oils such as sunflower oil or fish-liver oil. Preparations can be effected both as dry and as wet granules. For parenteral administration (subcutaneous, intravenous, intra-arterial, or intramuscular injection), the compositions or their physiologically tolerated derivatives such as salts, esters, N-oxides, and the like are converted into a solution, suspension, or emulsion, if desired, with the substances customary and suitable for this purpose, for example, solubilizers or other auxiliaries. Examples include: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, if desired, the composition can contain minor amounts of auxiliary substances such as wetting or emulsifying agents, or pH buffering agents that enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule) which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the inhibitory nucleic acids and their prodrugs or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer (1990). Science, 249:1527-1533; Treat et al. (1989). in Lopez-Berestein and Fidler (eds.), Liposomes in the Therapy of Infectious Disease and Cancer, Liss, N.Y., pp. 353-365).

Suitable salts of the compositions disclosed herein include pharmaceutically acceptable salts. Other salts, however, may be useful in the preparation of the compounds according to the present disclosure or of their pharmaceutically acceptable salts. Suitable pharmaceutically acceptable salts of the compounds of this disclosure include acid addition salts which may, for example, be formed by mixing a solution of the compound according to this disclosure with a solution of a pharmaceutically acceptable acid such as hydrochloric acid, sulphuric acid, methanesulphonic acids fumaric acid, maleic acid, succinic acid, acetic acid, benzoic acid, oxalic acid, citric acid, tartaric acid, carbonic acid or phosphoric acid.

Embodiments of the present disclosure include compositions and methods for treating viral pathogens. In one embodiment, the viral pathogens are treated by inactivation, inhibition, and/or destruction, without destruction of other healthy cells or tissue within a host organism. One exemplary embodiment provides fluid compositions that are delivered in any of the methods discussed above. It has been discovered that compositions having an active ingredient comprising a quaternary ammonium salt and other optional agents are effective in reducing, controlling, abating, inactivating, or eliminating viral pathogens. Preferably, the active ingredient or ingredients of the disclosed compositions are classified by the United States Food and Drug Administration as over-the-counter substances.

Another embodiment provides compositions having an active agent consisting essentially of a quaternary ammonium salt in an amount effective to reduce, inhibit, destroy, or inactivate viral pathogens, in combination with a pharmaceutically acceptable carrier. The pharmaceutically acceptable carrier can be suitable for intranasal or intrapulmonary delivery. Suitable quaternary ammonium salts have the following formula: 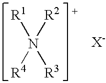
wherein N has a valency of 5; R¹, R², R³, R⁴ are the same or different and are independently selected from H, an alkyl group, an alkoxy group, a cycloalkyl group, a heterocycloalkyl group, an aryl group, a heteroaryl group, an acyl group, or a thioacyl group; and X is an anion, preferably a halogen.

Representative quaternary ammonium compounds can be divided into the following general categories:

(1) monoalkyltrimethyl ammonium salts such as cetyltrimethylammonium bromide (CTAB);

(2) monoalkyldimethylbenzyl ammonium salts such as benzalkonium chloride;

(3) dialkyldimethyl ammonium salts; and

(4) heterocyclic ammonium salts, for example when R¹ is an alkyl chain C₈-C₁₈ and remaining R², R³, and R⁴ groups are bridged to form an aromatic ring, for example pyridine in cetylpyridinium chloride. Thus, representative compounds include pyridinium quaternary salts, particularly substituted pyridinium quaternary salts such as lapirium chloride; and

(5) bisquaternary ammonium salts such as 4-aminoquinaldinium derivatives, dequalinium chloride, and hedquinium chloride.

Representative ammonium compounds also include ipratropium bromide, hyoscine butylbromide, mepenzolate bromide, pipenzolate bromide, poldine methylsulphate, propantheline bromide, cetrimide, methylbenzethonium chloride, benzethonium chloride, cetalkonium chloride, dofanium chloride, and domiphen bromide. The disclosed compositions can have at least one quaternary ammonium salt or combinations of multiple quaternary ammonium salts.

The active agent can also include chlorhexidine and other diguanides, such as for example, chlorhexidine gluconate and/or chlorhexidine acetate.

One or more flavoring agents may be added to the disclosed compositions. The flavoring agent can include natural or artificial flavors including natural or artificial sweeteners. Flavoring agents include, but are not limited to, any fruit flavor such as berry flavors, apple, cherry, plum, raisin, banana, pear, peach, figs, dates, lemon, coconut, and so on. Flavoring agents can also include any nut flavors as well as any sweet flavors such as chocolate, vanilla, caramel, butterscotch, cinnamon, graham flavors, mint, and so on. Flavoring agents additionally include any savory flavors such as all meat, game, fowl, fish, dairy, barbecue, smoke, pepper, and vegetable flavors.

The compositions also can include a carrier, for example a pharmaceutically acceptable carrier. Often the pharmaceutically acceptable carrier is an aqueous pH buffered solution. Examples of pharmaceutically acceptable carriers include buffers such as phosphate, borate, citrate and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. In certain embodiments, the carrier is also suitable for intranasal delivery and can include water or a mild or dilute saline solution, preferably a physiologically balanced saline solution. Additionally, the ion concentration of the carrier can be adjusted to provide a mild antibacterial effect. Saline solutions are also commonly used as moisturizers at present.

In another embodiment, the control and/or elimination of viral pathogens is accomplished by delivering the disclosed compositions to an infected organism, or treating infected biological source material with the disclosed compositions. The active agent of the compositions assists in the inhibition, destruction, or inactivation of viral pathogens.

Anti-Microbials

In yet another embodiment, the compositions contain an effective amount of an anti-microbial, for example alcohol, to provide an antibacterial effect. The alcohol is not necessary, but may facilitate mixing of the other components. Additionally, preservatives, mucolytic agents, anti-inflammatory agents, anti-histamines, desensitizing agents, or combinations thereof may be added to the compositions as needed.

Another embodiment provides compositions and methods optionally including an anti-microbial agent such as an antibiotic. Suitable antibiotics include beta-lactams such as natural and artificial penicillins and cephalosporins. Representative beta-lactams include, but are not limited to, penicillin G, and cephalothin. Semisynthetic penicillins include, but are not limited to, ampicillin, amoxycillin, and methicillin. Clavulanic acid can also be used either alone or in combination with another antibiotic such as amoxycillin sold under the mark Augmentin®.

Monobactams such as aztreonam can also be used with the disclosed compositions. Carboxypenems such as imipenem are also useful. The class of antibiotics known as aminoglycosides including streptomycin, gentamicin, kanamycin, and tobramycin are additional representative antibiotics.

Glycopeptides such as vancomycin, lincomycins such as clindamycin and macroclides such as erythromycin and oleandomycin can also be used with the disclosed compositions. Polypeptides including polymyxin and bacitracin, rifamycins, tetracyclines such as chlortetracycline and semisynthetic tetracycline such as doxycycline can also be used. Additional antibiotics include chloramphenicol, quinolones including nalidixic acid, sulfonamides such as gantrisin and trimethoprim. Finally, isoniazid (INH), paraaminosalicylic acid (PAS), and ethambutol can be used as anti-microbials.

Various embodiments of the composition exist in which various antiseptic and/or anti-microbial agents are used. In one embodiment of the composition, the antiseptic agent used is cetylpyridinium chloride (CPC). Other antiseptic and/or anti-microbial agents include, but are not limited to, chlorhexidine digluconate, hexetidine, sanguinanine, triclosan, and benzalkonium chloride. Still other antiseptic agents include ethanol (1-70%), isopropanol (1-70%), tincture of iodine (2% I2 in 70% alcohol), silver ions such as silver nitrate (AgNO₃), and mercuric chloride. It will be appreciated that one or more of these and other known antiseptics can be include in the disclosed compositions in an anti-microbially effective amount.

Desensitizing Agents

Another embodiment provides compositions having a desensitizing agent. The desensitizing agent is an agent that assists in preventing any allergic reactions due to delivery of the disclosed compositions. Representative desensitizing agents include local anesthetics or analgesics such as antipyrine, aspirin, benzocaine, benzyl alcohol, butamben picrate, dibucaine, dimethisoquine hydrochloride, dyclonine hydrochloride, lidocaine, methyl salicylate, phenacaine hydrochloride, phenolate sodium, pramoxine hydrochloride, pyrilamine maleate, resorcinol, salicyl alcohol, salicylamide, tetracaine, thymol, tripelenamine hydrochloride, trolamine salicylate, or combinations thereof.

Mucolytic Agents

The disclosed compositions can also include a mucolytic agent to assist in the breakup of mucous. Representative mucolytic agents include ammonium chloride, antimony potassium tartrate, benzoin tincture, calcium iodide, chloroform, guaifenesin, horehound, hydriodic syrup, iodized lime, ipecac, potassium guaiacolsulfonate, potassium iodide, sodium citrate, squill, terpin hydrate, tolu, and combinations thereof.

Surfactants

As noted, the disclosed compositions optionally include a surfactant. Preferred surfactants include anionic surfactants, cationic surfactants, non-ionic surfactants, zwitterionic surfactants, and mixtures thereof. Favorable surfactants include vitamin E polyethylene glycol 1000 succinate, polyoxyethylene sorbitan fatty acid esters, polyoxyethylene stearates, polyoxyethylene alkyl ethers, polyoxyethylene castor oils, polyglycolyzed glycerides, transesterified and (poly)ethoxylated oils, sorbitan fatty acid esters, poloxamers, fatty acids salts, bile salts, alkylsulfates, lecithins, mixed micelles of bile salts and lecithins, sugar esters, and mixtures thereof. Exemplary surfactants include sodium lauryl sulfate, sorbitan monolaurate, sorbitan monostearate, polyoxyethylene sorbitan monooleate, Polyoxyl 40 Stearate, Polyoxy ethylene 50 Stearate, and bile salts.

Antirhinoviral Agents

Zinc ions are powerful and natural antirhinoviral agents, immune system aids, interferon inducers, cell plasma/membrane pore closing agents, anti-inflammatory agents, antioxidants, protease inhibitors, and strong drying agents. It has been found that zinc ion availability (ZIA) values of approximately 100 will shorten the common cold by an average of seven days. Prior to the composition of this disclosure, ZIA 100 was only available in the form of zinc acetate lozenges. The composition of this disclosure can also incorporate the ZIA 100 zinc acetate, or zinc chloride, thereby providing further healing and soothing properties to the composition. The zinc acetate or zinc chloride provided in the composition is pleasant tasting, flavor stable and causes no objectionable after taste.

### EXAMPLE 1

The following formula provides a first representative example of an aqueous solution of the composition.

| Ingredient | Amount |
|---|---|
| Sodium chloride | 0.65% |
| Methyl salicylate | 0.02% |
| Benzalkonium chloride | 0.002%-0.015% |
| PEG or glycerin | Trace (Optional) |
| Zinc acetate/zinc chloride | Optional |
| Thimerosal | 0.001% |

Based on experiments that have been conducted, the methyl salicylate and menthol can be replaced by other similar acting ingredients to completely change the flavor. The base ingredients of the composition, preservative(s) and alcohol, are in percentage amounts that will remain relatively constant. The solution is prepared according to known techniques and excipients, as described in “Remington's Pharmaceutical Sciences Handbook,” 17^{th} ed., Hack Publ. Co., N.Y., U.S.A.

### EXAMPLE 2

The following formula provides a second representative example of an isotonic, sterile, aqueous solution of the composition. In this formula, the function of the ingredients is given under “Description”.

| | | | Percentage |
|---|---|---|---|
| Range | Agent | Description | (W/W) |
| 0.1 to 2% | Sodium chloride | Osmotic | 0.650 |
| | | agent | |
| 0.1 to 5% | Sodium borate | Buffering | 0.100 |
| | | agent | |
| 0.1 to 9% | Alcohol SD | Solubilizing | 0.090 |
| | | agent | |
| 0.001 to 2% | Edetate disodium | Preservative | 0.050 |
| 0.1 to 3% | Glycerin | Solubilizing | 0.001 |
| | | agent | |
| 0.001 to 5% | Poloxamer 407 | Antiseptic | 0.030 |
| 0.001 to 5% | Domiphen bromide | Antiseptic | 0.030 |
| 0.001 to 5% | Cetylpyridinium | Anti- | 0.040 |
| | chloride | infective | |
| 0.1 to 2% | Sorbitol | Sweetener | 0.002 |
| 0.1 to 2% | Sodium saccharin | Sweetener | 0.002 |
| 0.1 to 5% | anesthetic | desensitizing | 0.005 |
| | | agent | |
| 0.001 to 5% | Zinc acetate/ | Healing | 0.040 |
| | zinc chloride | agent | |
| | Deionized water | Solvent | <100% |
| | | (vehicle) | |
| 0.001 to 5% | Polysorbate 80 | Surfactant | 0.045 |

3. Methods of Treatment and Use

Other embodiments of the present disclosure provide methods of treating pathologies, for example viral infections, in a host by administering to a host an effective amount of a quaternary ammonium composition, for example cetylpyridinium chloride. The inhibitory agent is in an amount sufficient to reduce, inhibit, or inactivate a virus.

The above-referenced pharmaceutical compositions can be used in methods of treating a host organism for a viral infections. For example, one method includes administering to the host an effective amount of a composition that includes at least one quaternary ammonium salt compound.

Further, the above-referenced pharmaceutical compositions can be used in methods of inhibiting, destroying, and/or inactivating viral contaminants in a biological source material. For example, one method includes contacting the biological host material with a quaternary ammonium salt compound.

The quaternary ammonium salt used in the methods can be any of the types or specific quaternary ammonium salts disclosed herein. Further, the compositions used in the methods can include any of the additional ingredients or excipients disclosed herein.

4. Assessment of Virucidal Efficacy of Disclosed Compositions

Cetylpyridinium chloride (CPC), or 1-hexa-decyl pyridinium chloride, is a quaternary nitrogenous compound with antimicrobial activity. The chemical structure of the compound is set forth below:

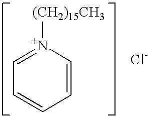

The compound is classified as a cationic surface-active agent and contains a cetyl radical on position 1 that renders molecules lypophilic, an attribute used for the antimicrobial activity. CPC, like chlorhexidine and hexetidine, is among the few cationic antiseptics that are commercially available as mouth rinse preparations. In addition, CPC is also commercially available as a nasal antiseptic spray under the registered trademark SINOFRESH® from SinoFresh HealthCare, Inc. of Englewood, Fla., US. The SinoFresh® product further includes the following in its formulation: benzalkonium chloride, dibasic sodium phosphate, eucalyptus oil, monobasic sodium phosphate, peppermint oil, polysorbate 80, propylene glycol, purified water, sodium chloride, sorbitol solution, spearmint oil, and wintergreen oil.

We examined the ability of the nasal spray formulation SINOFRESH® Nasal, Oral, & Sinus Care product and CPC at 0.05% (the concentration at which the active ingredient is present in the SinoFresh® nasal spray) to inhibit the infectivity of two major human respiratory viruses: Adenovirus (Ad), a double-stranded DNA, nonenveloped virus; and RSV, a single-stranded RNA, enveloped virus. The experiments included human (hAd) serotypes of species B (hAd3), C (hAd5), and E (hAd4) and the long strain of RSV, representative of A. Two independent experiments were carried out for each virus.

Ad3p (strain GB), Ad4p (strain RI-67), and Ad5p (strain 169) were pretreated with 1 volume of CPC 0.10% in Hanks or 1 volume of SinoFresh® 2× CPC (final concentration 0.10%) for 1 h at 35° C. Phosphate buffered saline (PBS) pretreatment of virus suspensions was used as a control.

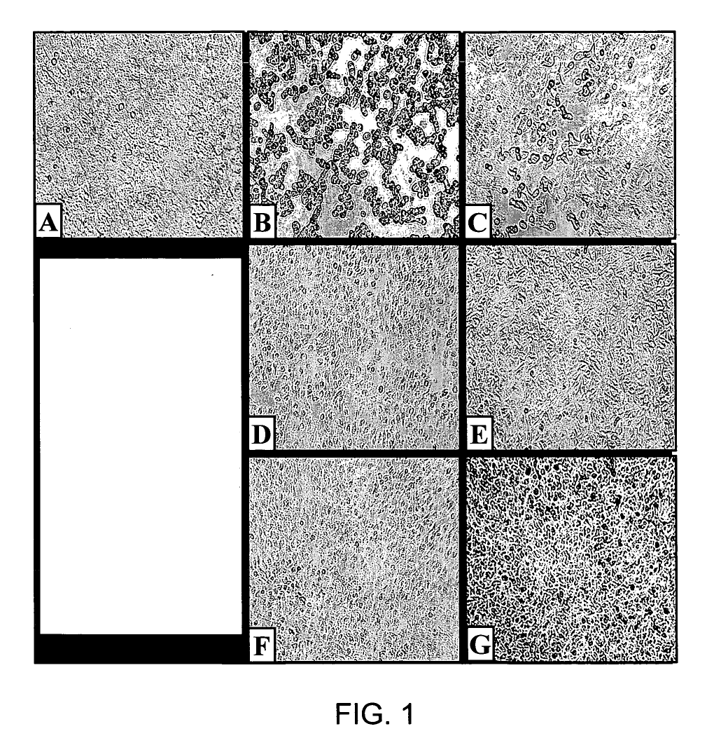

After the 1-h incubation, 100 μl of the treated virus containing a total of 10⁷ to 10⁶ plaque forming units (PFU) of virus or their corresponding 1:10 or 1:100 dilutions were used to infect monolayers of A549 lung epithelial cells in quadruplicate 24-well plates. Cells were observed for the development of cytopathic effect (CPE) over one week. As shown in FIG. 1, infection with PBS-treated hAd4 resulted in a marked CPE at 2 days post infection, whereas cell monolayers challenged with either SinoFresh® product-treated or CPC-treated virus developed no obvious CPE over the same period of time. FIG. 1 demonstrates the reduction of hAd4 infectivity in A549 human lung epithelial cells. A549 cell monolayers in 24-well plates were infected with 10⁷ PFU of hAd4 (RI-67strain) pretreated for 1 h at 35° C. with PBS (B); SinoFresh® product (D); or CPC (F). Viral cytopathic effect was examined at 2 days post infection. One hundred μl of a dilution 1:10 of the original treated samples containing 10⁶ PFU were used to infect additional wells. (C: PBS-treated 1:10; E: SinoFresh® product-treated 1:10; G: CPC-treated 1:10). Block A shows the uninfected control monolayer.

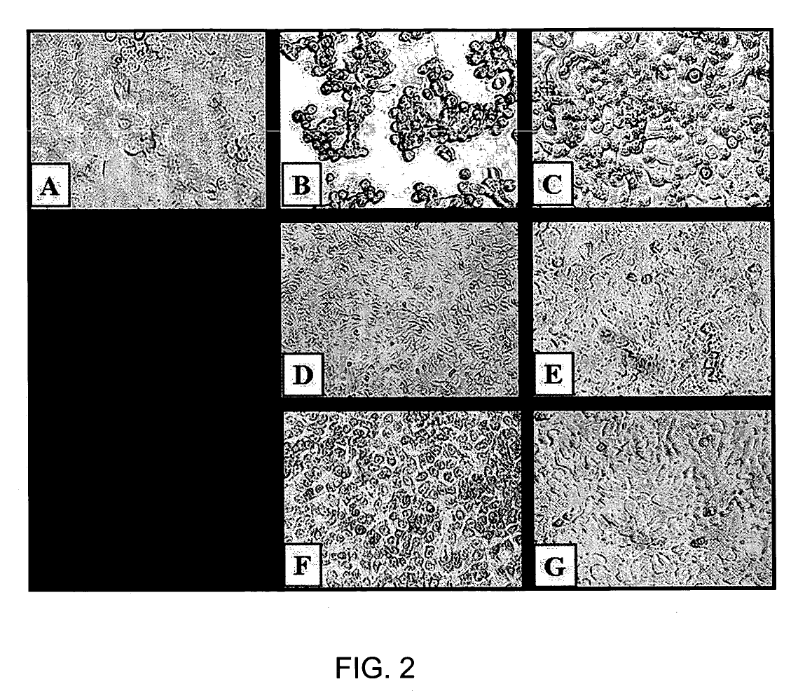

FIG. 2 shows the results obtained after pretreatment of hAdS following an identical protocol. In particular, FIG. 2 demonstrates the reduction of hAd4 infectivity in A549 human lung epithelial cells. A549 cell monolayers in 24-well plates were infected with 10⁶ PFU of hAd5 (169 strain) pretreated for 1 h at 35° C. with PBS (B); Sinofresh® product (D); or CPC (F). Viral cytopathic effect was examined at 3 days post infection. One hundred μl of a dilution 1:100 of the original treated samples containing 10⁴ PFU were used to infect additional wells. (C: PBS-treated 1:100; E: Sinofresh® product-treated 1:100; G: CPC-treated 1:100). Block A shows the uninfected control cell monolayer. Similar results were obtained with hAd3 (data not shown).

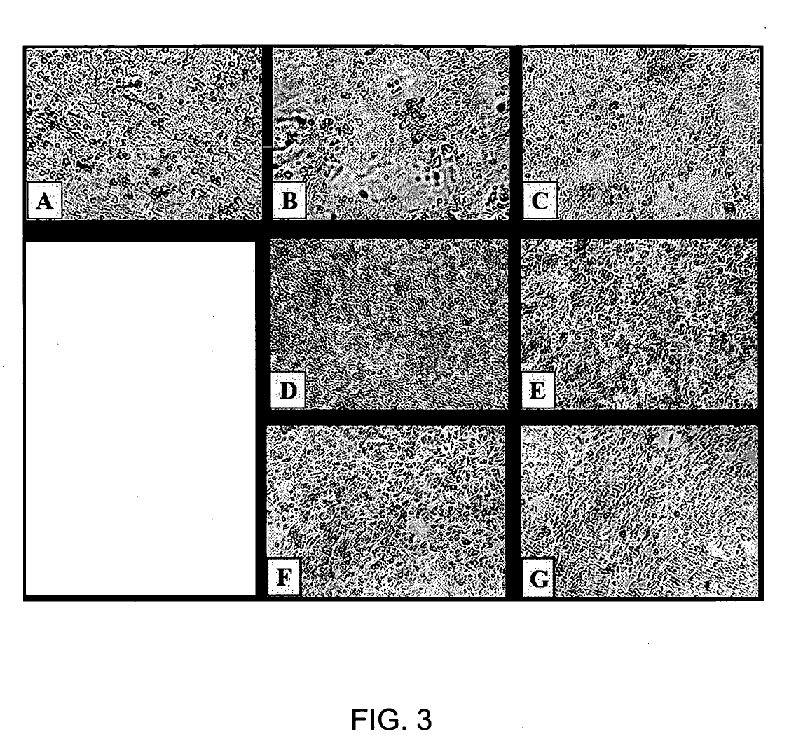

FIG. 3 shows the results of the experiments carried out with RSV. In the first experiment, a total of 10⁷ PFU were pretreated with 1 volume of PBS, CPC 0.10% in Hanks, or SinoFresh® product 2× CPC (final concentration 0.10%) for 1 h at 35° C. One hundred microliters (μl) of each virus suspension containing 5×106 PFU or 5×105 PFU were subsequently used to infect monolayers of Hep-2 cells in quadruplicate in 24-well plates. In particular, the protocol was as follows: Hep-2 cell monolayers in 24-well plates were infected with 5×10⁷ PFU of RSV (Long strain) and pretreated for 1 h at 35° C. with PBS (B); SinoFresh® product (D) or CPC (F). Viral cytopathic effect was examined at 2 days post infection. One hundred μl of a dilution 1:10 of the original treated samples containing 5×10⁶ PFU were used to infect additional wells (Block C: PBS-treated 1:10; E: SinoFresh® product-treated 1:10; G: CPC-treated 1:10). Block A shows the uninfected control monolayer.

A second independent experiment was carried out with a starting concentration of 109 PFU. In both experiments pretreatment with either SinoFresh® product or 0.05% CPC prevented syncytia formation in infected monolayers.

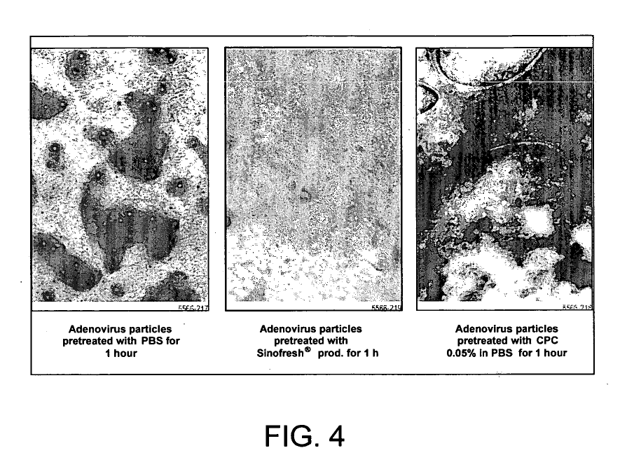

A suspension of hAd4 viral particles was treated with PBS, CPC 0,05% in PBS or SinoFresh™ for 1 h at room temperature. A total disruption of viral particles was observed, as shown in FIG. 4. Shown there is the electron microscopy of hAd4 viral particles treated with PBS, CPC 0.05% in PBS or SinoFresh® product for 1 h at room temperature.

The experiments show strong evidence that SinoFresh® product and its active ingredient, CPC, can reduce the infectivity of highly infectious doses of respiratory viruses representing the two major structural groups of enveloped and nonenveloped particles with DNA or RNA genomes. After exposure to the product, adenoviral particles are disrupted. Preliminary data and previous reports of antimicrobial activity in vivo suggest that these products will also be active and effective in primary human epithelial cells and also in animal models of acute viral infection.

It should be emphasized that the above-described embodiments, particularly any “preferred” embodiments, are merely possible examples of implementations, and are merely set forth for a clear understanding of the principles set forth herein. Many variations and modifications may be made to the above-described embodiment(s) of the compositions and methods without departing substantially from the spirit and principles of the disclosure. All such modifications and variations are intended to be included herein within the scope of this disclosure and protected by the following claims.

We claim:

1. A pharmaceutical composition for the treatment of viral infections comprising a quaternary ammonium salt compound.

2. The composition of claim 1, wherein the composition further comprises:
a pharmaceutically acceptable carrier.

3. The composition of claim 1, wherein the quaternary ammonium salt compound is chosen from at least one of the following:
monoalkyltrimethyl ammonium salts;
monoalkyldimethylbenzyl ammonium salts;
dialkyldimethyl ammonium salts;
heterocyclic ammonium salts;
pyridinium quaternary salts;
substituted pyridinium quaternary salts; and
bisquaternary ammonium salts.

4. The composition of claim 3, wherein the heterocyclic ammonium salt includes an alkyl chain C₈-C₁₈ and other alkyl groups bridged to form an aromatic ring.

5. The composition of claim 1, wherein the quaternary ammonium salt compound is chosen from at least one of the following: cetyltrimethylammonium bromide (CTAB), benzalkonium chloride, pyridine in cetylpyridinium chloride, lapirium chloride, 4-aminoquinaldinium derivatives, dequalinium chloride, and hedquinium chloride.

6. A method of treating a host organism for a viral infection comprising administering to the host an effective amount of a composition comprising an quaternary ammonium salt compound.

7. The method of claim 6, wherein the composition further comprises:
a pharmaceutically acceptable carrier.

8. The method of claim 6, wherein the quaternary ammonium salt compound is chosen from at least one of the following:
monoalkyltrimethyl ammonium salts;
monoalkyldimethylbenzyl ammonium salts;
dialkyldimethyl ammonium salts;
heterocyclic ammonium salts;
pyridinium quaternary salts;
substituted pyridinium quaternary salts; and
bisquaternary ammonium salts.

9. The method of claim 8, wherein the heterocyclic ammonium salt includes an alkyl chain C₈-C₁₈ and other alkyl groups bridged to form an aromatic ring.

10. The method of claim 6, wherein the quaternary ammonium salt compound is chosen from at least one of the following: cetyltrimethylammonium bromide (CTAB), benzalkonium chloride, pyridine in cetylpyridinium chloride, lapirium chloride, 4-aminoquinaldinium derivatives, dequalinium chloride, and hedquinium chloride.

11. A method of inhibiting, destroying, and/or inactivating viral contaminants in a biological source material comprising contacting the biological host material with a quaternary ammonium salt compound.

12. The method of claim 11, wherein the composition further comprises:
a pharmaceutically acceptable carrier.

13. The method of claim 11, wherein the quaternary ammonium salt compound is chosen from at least one of the following:
monoalkyltrimethyl ammonium salts;
monoalkyldimethylbenzyl ammonium salts;
dialkyldimethyl ammonium salts;
heterocyclic ammonium salts;
pyridinium quaternary salts;
substituted pyridinium quaternary salts; and
bisquaternary ammonium salts.

14. The method of claim 13, wherein the heterocyclic ammonium salt includes an alkyl chain C₈-C₁₈ and other alkyl groups bridged to form an aromatic ring.

15. The method of claim 1 1, wherein the quaternary ammonium salt compound is chosen from at least one of the following: cetyltrimethylammonium bromide (CTAB), benzalkonium chloride, pyridine in cetylpyridinium chloride, lapirium chloride, 4-aminoquinaldinium derivatives, dequalinium chloride, and hedquinium chloride.
